(19) United States
(12) Patent Application Publication
(10) Pub. No.: US 2016/0030232 A1
(43) Pub. Date: Feb. 4, 2016

(54) DENTAL RETAINER SYSTEM

(71) Applicants: Jiahua ZHU, San Francisco, CA (US); Hilary M. ZHU, San Francisco, CA (US)

(72) Inventors: Jiahua ZHU, San Francisco, CA (US); Hilary M. ZHU, San Francisco, CA (US)

(21) Appl. No.: 14/817,693

(22) Filed: Aug. 4, 2015

Related U.S. Application Data
(60) Provisional application No. 62/033,055, filed on Aug. 4, 2014.

(51) Int. Cl. A61F 5/56; A61B 5/00; A61C 7/16

(52) U.S. Cl. CPC A61F 5/566; A61C 7/16; A61B 5/4557; A61F 2005/563

(57) ABSTRACT

Systems and methods for securing a person's upper jaw in place relative to the lower jaw. Included are dental mounts affixed to teeth on the upper and lower jaw as well as a support affixed between the mounts to hold the jaw in place. Some embodiments include an extended support having a substantially U-shaped portion for protecting teeth from injury.

### PRIORITY

This application claims the benefit of co-pending U.S. provisional application No. 62/033,055 entitled Dental Retainer System, filed Aug. 4, 2014 by the same inventors which is included by reference as if fully set forth herein.

### BACKGROUND

Bruxism is the excessive grinding of the teeth and/or excessive clenching of the jaw. It is an oral parafunctional activity in that it is unrelated to normal jaw functions such as eating or talking. Bruxism is a common problem: reports of prevalence range from 8-31% in the general population. Several symptoms are commonly associated with bruxism, including hypersensitive teeth, aching jaw muscles, and headaches. Bruxism may cause tooth wear, or damage to teeth and dental restorations.

There are two main types of bruxism: that which occurs during sleep (sleep bruxism) and that which occurs during wakefulness (awake bruxism). Dental damage may be similar in both types, but the symptoms of sleep bruxism tend to be worst on waking and improve during the course of the day, and the symptoms of awake bruxism may not be present at all on waking, and then worsen over the day. The causes of bruxism are not completely understood, but probably involve multiple factors. Occlusal splints (also termed dental guards) are commonly prescribed, mainly by dentists, as a treatment for bruxism. Splints may also reduce muscle strain by allowing the upper and lower jaw to move easily with respect to each other. A dental guard is typically worn during every night's sleep on a long-term basis.

Conventional occlusal splints are available as either partial or full-coverage splints according to whether they fit over some or all of the teeth. They are typically made of plastic (e.g. acrylic) and can be hard or soft. A lower appliance can be worn alone, or in combination with an upper appliance. A known problem with wearing a splint can be stimulation of salivary flow, and for this reason some advise to start wearing the splint about 30 minutes before going to bed so this does not lead to difficulty falling asleep. Another disadvantage for hypersensitive teeth in bruxism is the need for a desensitizing tooth paste (e.g. containing strontium chloride) may also be applied initially inside the splint so the material is in contact with the teeth all night.

### SUMMARY

Disclosed herein are systems and methods for securing a person's upper jaw in place relative to the lower jaw. Included are dental mounts affixed to teeth on the upper and lower jaw as well as a support affixed between the mounts to hold the jaw in place. Some embodiments include an extended support having a substantially U-shaped portion for protecting teeth from injury. The disclosure herein does not require a conventional retainer because this disclosure does not cover any teeth. Accordingly the problems associated with conventional retainers are obviated.

The construction and method of operation of the invention, however, together with additional objectives and advantages thereof will be best understood from the following description of specific embodiments when read in connection with the accompanying drawings.

### BRIEF DESCRIPTION OF THE DRAWINGS

FIG. 1 shows a support strip according to certain aspects of the current disclosure.

FIG. 2 shows a dental mount.

FIG. 3 shows an embodiment with a guard mounted on teeth and positioned to guard the teeth.

### DESCRIPTION

### Generality of Invention

This application should be read in the most general possible form. This includes, without limitation, the following:

References to specific techniques include alternative and more general techniques, especially when discussing aspects of the invention, or how the invention might be made or used.

References to “preferred” techniques generally mean that the inventor contemplates using those techniques, and thinks they are best for the intended application. This does not exclude other techniques for the invention, and does not mean that those techniques are necessarily essential or would be preferred in all circumstances.

References to contemplated causes and effects for some implementations do not preclude other causes or effects that might occur in other implementations.

References to reasons for using particular techniques do not preclude other reasons or techniques, even if completely contrary, where circumstances would indicate that the stated reasons or techniques are not as applicable.

Furthermore, the invention is in no way limited to the specifics of any particular embodiments and examples disclosed herein. Many other variations are possible which remain within the content, scope and spirit of the invention, and these variations would become clear to those skilled in the art after perusal of this application.

### Lexicography

The terms “effect”, “with the effect of” (and similar terms and phrases) generally indicate any consequence, whether assured, probable, or merely possible, of a stated arrangement, cause, method, or technique, without any implication that an effect or a connection between cause and effect are intentional or purposive.

The term “relatively” (and similar terms and phrases) generally indicates any relationship in which a comparison is possible, including without limitation “relatively less”, “relatively more”, and the like. In the context of the invention, where a measure or value is indicated to have a relationship “relatively”, that relationship need not be precise, need not be well-defined, need not be by comparison with any particular or specific other measure or value. For example and without limitation, in cases in which a measure or value is “relatively increased” or “relatively more”, that comparison need not be with respect to any known measure or value, but might be with respect to a measure or value held by that measurement or value at another place or time.

The term “substantially” (and similar terms and phrases) generally indicates any case or circumstance in which a determination, measure, value, or otherwise, is equal, equivalent, nearly equal, nearly equivalent, or approximately, what the measure or value is recited. The terms “substantially all” and “substantially none” (and similar terms and phrases) generally indicate any case or circumstance in which all but a relatively minor amount or number (for “substantially all”) or none but a relatively minor amount or number (for “substantially none”) have the stated property. The terms “substantial effect” (and similar terms and phrases) generally indicate any case or circumstance in which an effect might be detected or determined.

The terms “this application”, “this description” (and similar terms and phrases) generally indicate any material shown or suggested by any portions of this application, individually or collectively, and include all reasonable conclusions that might be drawn by those skilled in the art when this application is reviewed, even if those conclusions would not have been apparent at the time this application is originally filed.

### DETAILED DESCRIPTION

Specific examples of components and arrangements are described below to simplify the present disclosure. These are, of course, merely examples and are not intended to be limiting. In addition, the present disclosure may repeat reference numerals and/or letters in the various examples. This repetition is for the purpose of simplicity and clarity and does not in itself dictate a relationship between the various embodiments and/or configurations discussed.

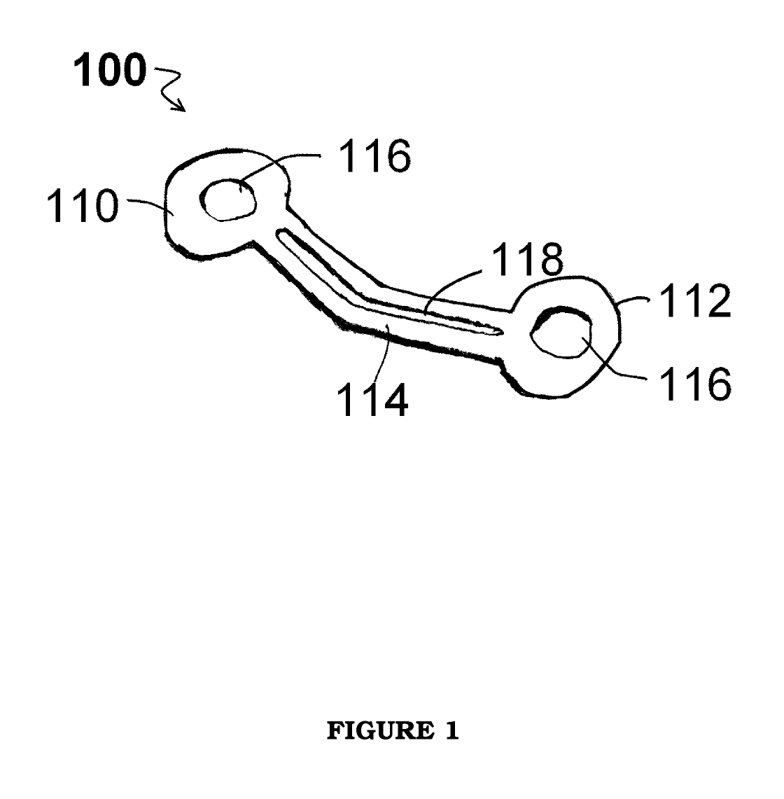

FIG. 1 shows a support strip 100 according to certain aspects of the current disclosure. The support strip includes two generally circular ends 110 and 112 with a main body 114. Each end 110 and 112 includes a mount hole 116 through the support strip 100 and certain embodiments may include a body hole 118 through the main body of the support strip 100.

The support strip 100 may be formed from plastics or resins or other suitable material. Structurally the support strips are rigid or semi-rigid depending on the application. Some embodiments of the support strip may have a straight body, while alternative embodiments may has an S-shaped or nonlinear shape. The body shape may be application dependent.

References in the specification to “one embodiment”, “an embodiment”, “an example embodiment”, etc., indicate that the embodiment described may include a particular feature, structure or characteristic, but every embodiment may not necessarily include the particular feature, structure or characteristic. Moreover, such phrases are not necessarily referring to the same embodiment. Further, when a particular feature, structure or characteristic is described in connection with an embodiment, it is submitted that it is within the knowledge of one of ordinary skill in the art to effect such feature, structure or characteristic in connection with other embodiments whether or not explicitly described. Parts of the description are presented using terminology commonly employed by those of ordinary skill in the art to convey the substance of their work to others of ordinary skill in the art.

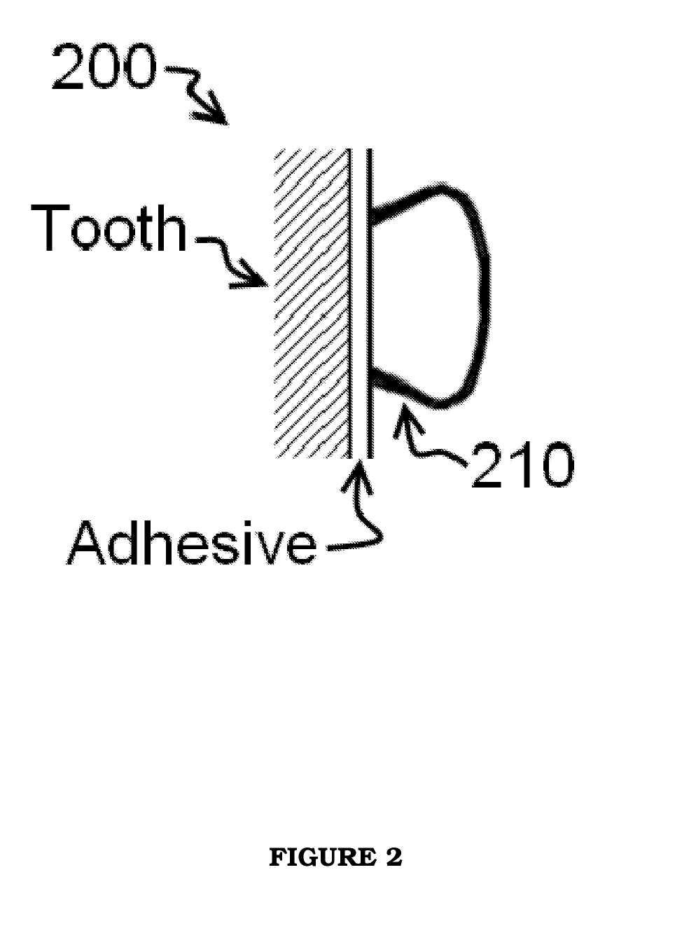

FIG. 2 shows a dental mount 200. The dental mount is a rigid or semi-rigid support shaped similarly to the shape of the mount hole 116 (supra). The dental mount 200 may also be formed from plastics, resins, or other suitable material. The dental mount 200 may include a lip or catch 210 similar to a bit, a bollard, a hook and the like, such that any fastener placed over the dental mount may be held in place. Some embodiments may include alternative shaped mounts to achieve certain aesthetic appeal or increased ease-of-use or functionality. These alternative shapes may include pointed sections to facilitate use.

In operation a dentist or other professional disposes the dental mounts 200 on a person's teeth. Affixing the dental mounts 200 may be effectuated using adhesives. Conventional dental adhesives including light-cured and self-cured adhesives among others. The type of adhesive employed may be directed by the dental problem being addressed by the Dental Retainer System.

The position of the dental mounts will depend on the nature of the bruxism or other dental malady being addressed. A first dental mount 200 may be secured to a tooth on the upper jaw, while a second dental mount 200 may be secured to a tooth on a lower jaw. Once the dental mounts are secured, a support strip 100 is affixed between the two dental mounts such that the mount holes 116 are placed over the dental mounts 200 and held in place by the lip on the dental mount.

The size and rigidity of the support strip 100 fixes the position of the teeth and also the upper and lower jaw. The shape of the support strip 100 and the location of the dental mounts allows for jaw positions to meet the patient's needs.

In certain embodiments, a user may create custom shaped support strips by determining the condition of a wearer of the device, then creating one or more support strips to address the condition, then affixing dental mounts at dental points to address the condition. Accordingly, there is no requirement that support strips be identical or even symmetrical.

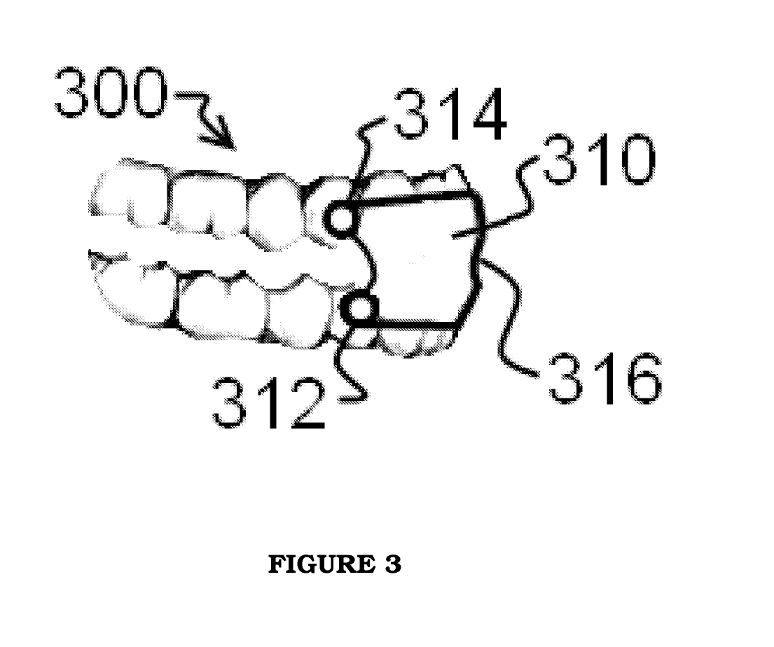

FIG. 3 shows an embodiment with a guard mounted on teeth and positioned to guard the teeth 300. The guard 300 may have mounting holes 312 and 314 for mounting the guard to dental mounts. The guard includes a support portion for supporting the attachment to dental mounts. Also included in the guard is a generally U-shaped tooth protection portion 310 (side view shown) positioned about the teeth in a manner to protect the teeth from damage should they suffer a blow.

In the embodiment of FIG. 3, the guard 300 may be positioned with the teeth open of closed together. In some embodiments an airway or passageway 316 may be formed in the guard to facilitate breathing or drinking. While FIG. 3 show as single embodiment of a guard 300, it is recognized that the guard may be effectuated in other shapes to accommodate different users and different conditions.

Exemplary embodiments are also included in the attached Appendix to the Specification which is included by reference as if fully set forth herein.

The above illustration provides many different embodiments or embodiments for implementing different features of the invention. Specific embodiments of components and processes are described to help clarify the invention. These are, of course, merely embodiments and are not intended to limit the invention from that described in the claims.

Although the invention is illustrated and described herein as embodied in one or more specific examples, it is nevertheless not intended to be limited to the details shown, since various modifications and structural changes may be made therein without departing from the spirit of the invention and within the scope and range of equivalents of the claims. Accordingly, it is appropriate that the appended claims be construed broadly and in a manner consistent with the scope of the invention, as set forth in the following claims.

What is claimed:

1. A device including:
a first substantially bollard-shaped dental mount;
a second substantially bollard-shaped dental mount, and
a semi-rigid support member, said support member including a hole for receiving the first dental mount and a hole for receiving the second dental mount.

2. The device of claim 1 wherein the support member includes a substantially U-shaped portion.

3. A method including:
disposing a first dental mount on a first tooth;
disposing a second dental mount on a second tooth;
disposing a rigid or semi-rigid support member on the first and second dental mount.

4. The method of claim 3 wherein the first and second dental mount are affixed to the first and second tooth with adhesive.

5. The method of claim 3 wherein the first tooth is a human tooth.

6. The method of claim 3 wherein the support member includes a substantially U-shaped portion.

7. The method of claim 3 further including:
diagnosing a medical condition, wherein said disposing of the first tooth and the second tooth operates to alleviate the medical condition.
